(19) United States
(12) Patent Application Publication
(10) Pub. No.: US 2012/0083680 A1
(43) Pub. Date: Apr. 5, 2012

(54) NEEDLE TIP AND NEEDLE PROBE

(76) Inventor: John Christopher Carr, Woking (GB)

(21) Appl. No.: 13/080,146

(22) Filed: Apr. 5, 2011

Related U.S. Application Data
(60) Provisional application No. 61/323,105, filed on Apr. 12, 2010.

(51) Int. Cl. A61B 5/0492; B24B 1/00; A61B 5/04

(52) U.S. Cl. 600/373; 451/49

(57) ABSTRACT

A monopolar needle probe for measurement of electrical activity in a patient. The needle probe has a nonconductive sheath and a conductive needle within the nonconductive sheath. The conductive needle has a longitudinal axis. An end of the needle is exposed from the nonconductive sheath and the end defines a plurality of substantially planar and symmetrical surfaces.

### CROSS-REFERENCE TO RELATED APPLICATIONS

This application is a non-provisional application which claims priority to U.S. provisional application Ser. No. 61/323,105, filed Apr. 12, 2010, which is incorporated by reference herein in its entirety.

### TECHNICAL FIELD

The invention relates generally to medical devices. More particularly, the invention pertains to a needle for use in a needle probe for measurement of electrical activity in a patient. The invention also relates to the tip geometry of such a needle. The invention relates particularly to a needle and needle probe suitable for use in electromyography (EMG).

### BACKGROUND

In EMG, the electrical activity of muscle tissue is recorded and interpreted. When a muscle fiber contracts, action potentials are generated. Signals from a given muscle, can be detected by use of an appropriately positioned electrode. This can be achieved by use of a needle probe inserted into the patient's tissue. The needle probe has a conductive inner core that terminates in a sharp tip. The needle probe also has a nonconductive outer sheath. The conductive tip is exposed from the nonconductive sheath for a controlled length, thus placing the conductive tip in contact with the patient's muscle tissue for a desired length. An external electrode placed on the skin adjacent to the needle probe is used as a signal reference to record electrical readings. Such a needle probe has been referred to as a monopolar EMG needle.

During an EMG procedure, the sharp tip of a needle probe is inserted into a patient. In the prior art, the industry standard monopolar needle uses a conical tip geometry. The conical tip geometry, however, is inherently not very sharp and, on occasion, present difficulties in penetrating the patient's tissue. Instead of a cutting process, a conical geometry tip can pierce the tissue, but then stretch the tissue as the needle is inserted, causing resistance to insertion.

In some prior art devices, a trocar tip geometry is used. The trocar tip, however, can present a complicated manufacturing process for coating the needle. Further, due to the lack of symmetry of a trocar geometry tip, readings from the probe may be irregular depending upon the orientation of the trocar geometry facets with respect to the external lead.

Thus, there is a need for a needle with improved sharpness that also exhibits reliable performance and is convenient to manufacture.

### BRIEF SUMMARY

In one embodiment, a monopolar needle probe for measurement of electrical activity in a patient is provided. The needle probe has a nonconductive sheath and a conductive needle within the nonconductive sheath. The conductive needle has a longitudinal axis. An end of the needle is exposed from the nonconductive sheath and the end defines a plurality of substantially planar and symmetrical surfaces.

In another embodiment, a method of manufacturing a monopolar needle probe is provided. In one step, a needle probe having a conductive needle within a nonconductive sheath is provided. In another step, a plurality of substantially planar and symmetrical surfaces is ground onto an end of the needle probe. The grinding removes at least a portion of the nonconductive sheath. After grinding one of the plurality of the substantially planar and symmetrical surfaces, another of the plurality of the substantially planar surfaces is ground after rotating the needle probe around a longitudinal axis of the needle probe.

Advantages of the present invention will become more apparent to those skilled in the art from the following description of the preferred embodiments of the invention which have been shown and described by way of illustration. As will be realized, the invention is capable of other and different embodiments, and its details are capable of modification in various respects. Accordingly, the drawings and description are to be regarded as illustrative in nature and not as restrictive.

### BRIEF DESCRIPTION OF THE DRAWINGS

FIG. 1 is a side view of a needle probe according to one embodiment of the present invention.

FIG. 2 is an end view of a needle probe according to one embodiment of the present invention.

FIG. 3 is a side view of a needle probe according to one embodiment of the present invention during a grinding process.

FIGS. 4A-4C depict test results showing, at least, sliding friction and penetration force involving three needle probe, including one according to an embodiment of the present invention.

FIG. 5 depicts a picture of the three needles used in the tests whose results are shown in FIGS. 4A-4C.

### DETAILED DESCRIPTION OF THE DRAWINGS AND THE PRESENTLY PREFERRED EMBODIMENTS

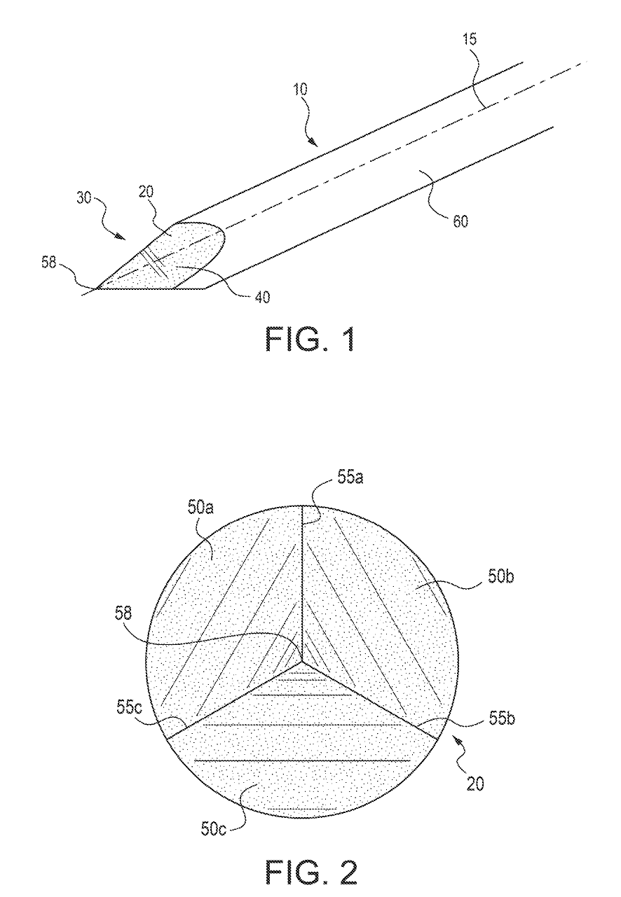

FIG. 1 depicts a needle probe 10 according to one embodiment of the present invention. The needle probe 10 defines a longitudinal axis 15 and comprises an electrically conductive needle 20 and an electrically nonconductive insulating sheath 60 for insulating the operator from the conductive needle 20. The needle 20 preferably is made from surgical grade stainless steel; however, other conductive and medically appropriate materials may be used. The sheath 60 preferably is made from polytetrafluoroethylene (PTFE); however, other nonconductive and medically appropriate materials may be used. The needle probe 10 defines a first end 30 for insertion into the patient. A tip 40 for cutting or piercing tissue is located at the first end 30 of the needle 20. The tip 40 is not covered by an insulating sheath 60 and, thus, an electrically conductive portion of the needle 20 is exposed at the tip 40.

With reference to the embodiment of FIGS. 1-2, the tip 40 defines three substantially planar and symmetrical surfaces 50a, 50b, 50c. In this embodiment, the surfaces 50a, 50b, 50c are at about 60 degree angles from each other. The intersection of the surfaces 50a, 50b, 50c form three cutting edges 55a, 55b, 55c. In this way, the needle 20 contains cutting edges not present in the conical tip geometry, thus reducing tissue stretching and the force required for insertion. Although three surfaces and cutting edges are shown in this embodiment, needle probes according to other embodiments may have additional surfaces and cutting edges. The angle between each of the surfaces 50a, 50b, 50c and the longitudinal axis 15 preferably is between about 5 degrees and about 20 degrees, more preferably is between about 10 degrees and about 18 degrees, and still more preferably is about 15 degrees. The surfaces 50a, 50b, 50c terminate in a common sharp point 58.

In one preferred embodiment, the exposed length of the needle 20 is between about 0.5 mm and about 1 mm, and more preferably, about 0.75 mm. In one preferred embodiment, the diameter of the needle 20 preferably is about 0.36 mm, an industry standard diameter for monopolar needles; however, a diameter of 0.46 mm or other diameter suitable for monopolar needles may also be used. In a variety of embodiments, the needle may further comprise an electrosurgical generator and be configured for use in an EMG procedure or other diagnostic or therapeutic procedure.

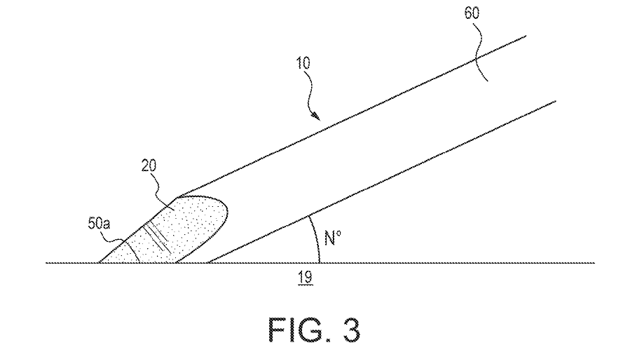

With reference to FIG. 3, in one embodiment, the needle probe 10 may be manufactured using a standard grinding wheel or other grinding/abrasion surface 19 and a needle probe 10 blank precoated with a nonconductive sheath 60. In this embodiment, the needle probe 10 blank is held at a constant angle N to the grinding wheel to form a surface, such as surface 50a. The angle N preferably is between about 5 degrees and about 20 degrees, more preferably is between about 10 degrees and about 18 degrees, and still more preferably is about 15 degrees. To form additional surfaces, the needle probe 10 conveniently needs only to be indexed around the longitudinal axis 15 for additional grinding. Thus, the symmetrical surfaces also provide a convenient method of manufacturing the needle probe 10. The grinding of a precoated needle probe 10 blank may also present an advantage of providing careful control over the exposure length of the conductive needle 20. The grinding process necessarily removes the insulating sheath 60 from the end 30 of the needle probe 10, exposing the conductive needle 20. The grinding process, such as the grind angle, can be carefully controlled to expose the desired exposure length. This method may also be suitable for scaling such that multiple needles can be ground at the same time.

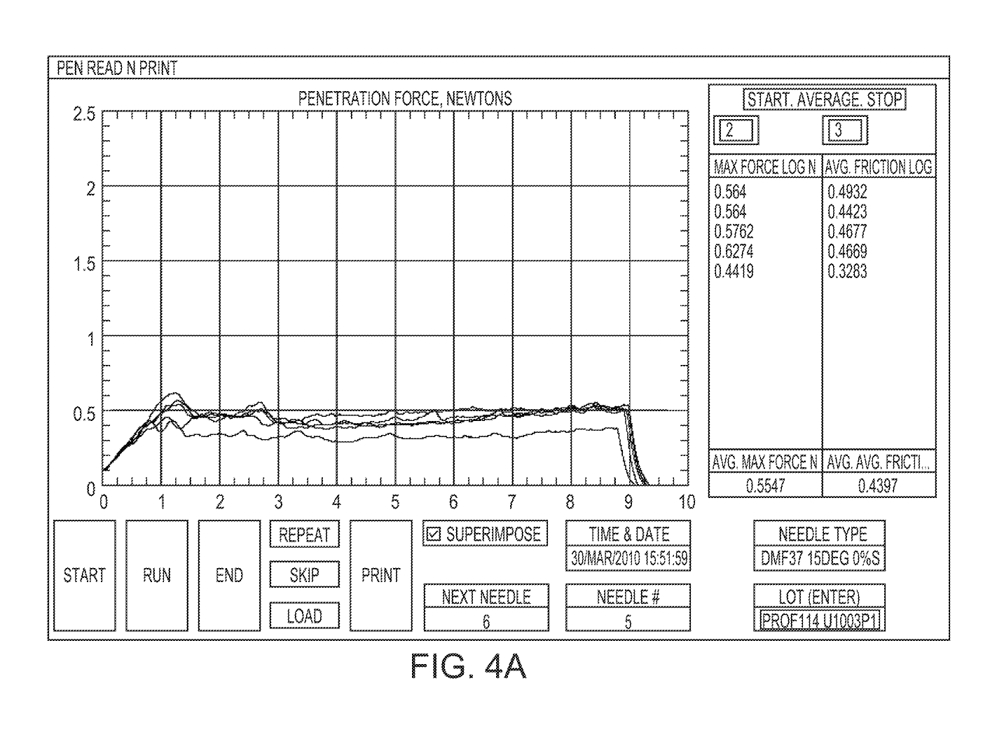

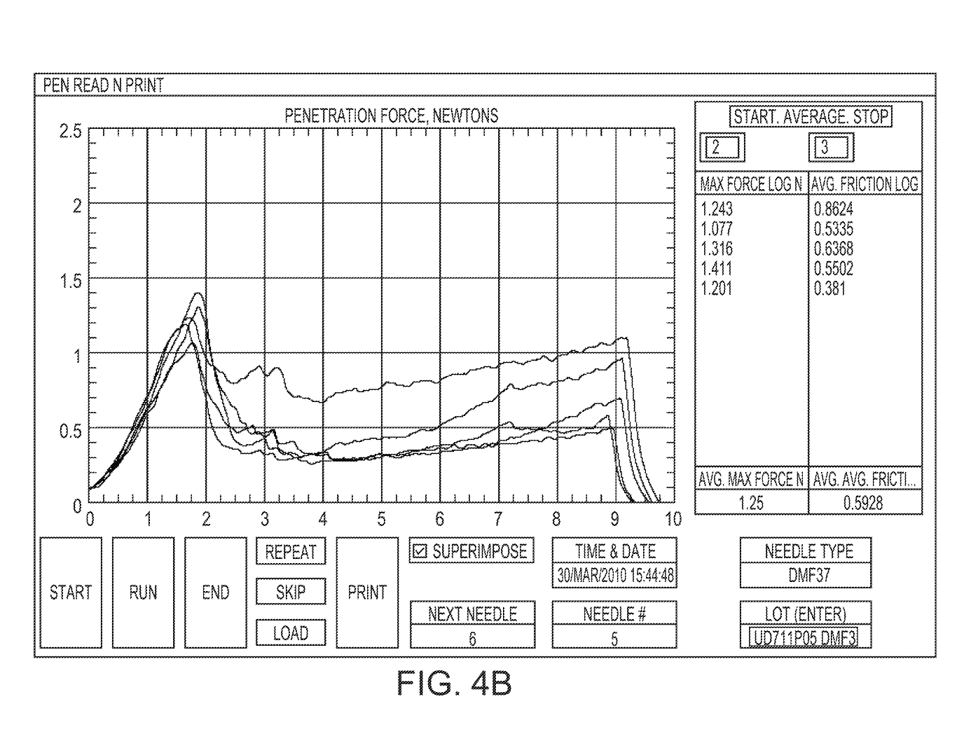

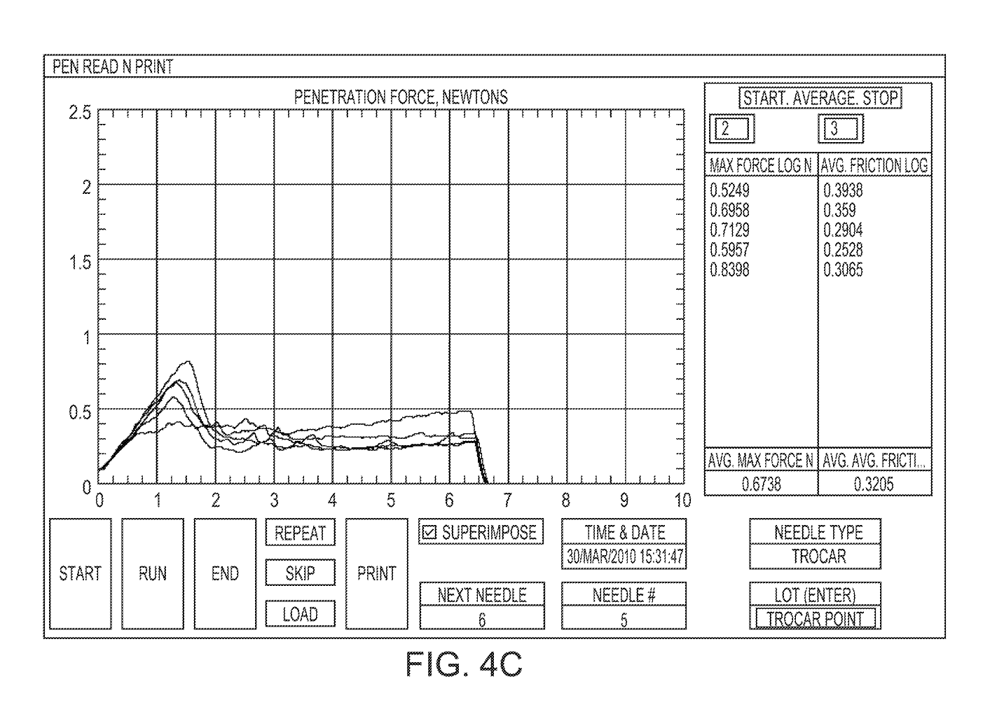

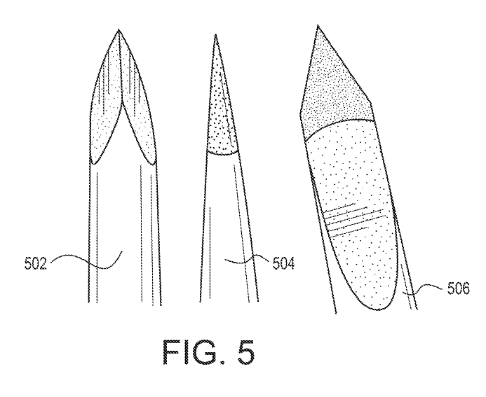

In operation, an external surface electrode (not shown) is attached to the exterior of the patient's skin adjacent to the tissue that is to be probed. The needle probe 10 is then inserted into the patient's tissue. The cutting edges 55a, 55b, 55c allow the operator to insert the needle with less force than the industry standard conical tips. Further, the symmetrical geometry may promote consistent readings regardless of the orientation of the probe. With reference to FIGS. 4A-4C and FIG. 5, graphs of test results are shown where the tests were conducted using a needle probe 502 constructed according to one embodiment of the present invention, a conical geometry tip needle 504, and a trocar geometry tip needle 506. Each needle was inserted five times into seven day old pork. The insertion sites were done in the same order so the traces could be compared. The penetration points at each site were within a few millimeters of each other to enable comparison.

As shown in FIGS. 4A-4C, the penetration force and sliding friction of the present needle design 502 compared favorably to the other two needles 504, 506. Specifically, FIG. 4A shows a graph of penetration and sliding force of a needle embodiment 502 constructed in keeping with the present invention. FIGS. 4B and 4C, respectively, show graphs of penetration and sliding force from tests using needles of generally the same dimensions but including a conical tip 504 and a trocar tip 506, with all three tips being shown in FIG. 5. The initial penetration force required by the present needle 502 was consistently lower, and the overall force profile is generally favorable as compared to the other tip designs.

It is therefore intended that the foregoing detailed description be regarded as illustrative rather than limiting, and that it be understood that it is the following claims, including all equivalents, that are intended to define the spirit and scope of this invention.

1. A monopolar needle probe configured for measurement of electrical activity in patient tissue, the needle probe comprising:
a nonconductive sheath
a conductive needle disposed substantially within the nonconductive sheath, the conductive needle having a longitudinal axis wherein an end of the needle is exposed from the nonconductive sheath and the exposed end defines a plurality of substantially planar and symmetrical surfaces that terminate at a common point.

2. The needle of claim 1 further comprising an electrosurgical generator and configured for use in an electromyography procedure.

3. The needle of claim 1 wherein the needle defines three substantially planar and symmetrical surfaces.

4. The needle of claim 1 wherein the needle defines four substantially planar and symmetrical surfaces.

5. The needle of claim 1 wherein the needle is exposed from the nonconductive sheath for between about 0.5 mm and about 1 mm.

6. The needle of claim 5 wherein the needle is exposed from the nonconductive sheath for about 0.75 mm.

7. The needle of claim 1 wherein the angle between the substantially planar and symmetrical surfaces and the longitudinal axis is between about 5 degrees and about 20 degrees.

8. The needle of claim 7 wherein the angle between the substantially planar and symmetrical surfaces and the longitudinal axis is between about 10 degrees and about 18 degrees.

9. The needle of claim 1 wherein the angle between the substantially planar and symmetrical surfaces and the longitudinal axis is about 15 degrees.

10. A method of manufacturing a monopolar needle probe comprising:
providing a needle probe comprising a conductive needle within a nonconductive sheath;
grinding a plurality of substantially planar and symmetrical surfaces on an end of the needle probe wherein the grinding removes at least a portion of the nonconductive sheath and wherein after grinding one of the plurality of the substantially planar and symmetrical surfaces, another of the plurality of the substantially planar surfaces is ground after rotating the needle probe around a longitudinal axis of the needle probe.

11. The method of claim 10 wherein three substantially planar and symmetrical surfaces are ground.

12. The method of claim 10 wherein four substantially planar and symmetrical surfaces are ground.

13. The method of claim 10 wherein the grinding is performed with a grinding wheel.

14. The method of claim 13 wherein the angle between the needle probe and the grinding wheel is between about 5 degrees and about 30 degrees.

15. The method of claim 14 wherein the angle between the needle probe and the grinding wheel is between about 10 degrees and about 20 degrees.

16. The method of claim 15 wherein the angle between the needle probe and the grinding wheel is about 15 degrees.
